(19) United States
(12) Patent Application Publication
(10) Pub. No.: US 2007/0148111 A1
(43) Pub. Date: Jun. 28, 2007

(54) Peel off synthetic body paint

(76) Inventor: Remy Simpson, Lexington, KY (US)

Correspondence Address: Michael Coblenz, 752 Rose Hurst Way, Lexington, KY, 40515, US

(21) Appl. No.: 11/314,572

(22) Filed: Dec. 22, 2005

(51) Int. Cl. A61K 8/73

(52) U.S. Cl. 424/63; 424/70.13

(57) ABSTRACT

A peel off synthetic body paint, and a method of making the peel off synthetic body paint, is disclosed. The peel off synthetic body paint is made from a combination of a non-toxic paint and a peel masque product. The non-toxic paint comprises a binder, a pigment, a diluent, and additives, and the peel masque product comprises plasticizer and water. The binder of the non-toxic paint is natural waxes, synthetic waxes, natural gums, synthetic gums, starch, gelatin, casein, or egg yolk. The plasticizer of the peel masque product is glycerine, propylene glycol, polyethylene glycol, or ethoxylated lanoline alcohol.

### CROSS REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

The present invention relates to body paint, or paint used to decorate or adorn the human body.

2. Description of the Related Art

Body paint is well known in a variety of arts, from the theater to children's entertainment to a variety of other recreational and entertainment uses.

Humans have adorned their bodies with decorations, both temporarily and permanently, since the beginning of time. Tattooing, which is the most common form of permanent adornment, has been practiced by humans since before recorded time. Tattoos come in all colors.

Use of henna, a dye obtained from the henna plant, for temporary body decoration has been practiced in the Middle East for many centuries. Traditional henna is a dark brown color, but modern henna can be in any color. Henna actually dyes the skin, and once applied it can remain for days or weeks.

Use of cosmetics and other colorations to alter or adorn the face or bodies of actors is also well known.

There has been a significant increase in the popularity of permanent tattooing in recent years. Because of the increase in permanent body adornment, there has also been a significant increased interest in, and demand for, temporary body art and body art products.

There are numerous commercial products for temporarily adorning the body with color. The two most common are colored cosmetics, also referred to as greasepaint, and body paints. Greasepaint is widely used in movies, the theatre, the circus, and other types of entertainment. Traditional greasepaint was literally made of grease colored with dyes. Greasepaint is easy to apply and remove. The ease of removal, however, creates problems because the coloration or decoration can be easily smeared or rubbed off.

One type of body paint is known as air-brush body paint. It is typically a cellulose based ink, and is used for air brush applied temporary tattoos. These are generally applied on small areas, and are generally designed to look like a permanent tattoo. The air-brush body paints are not designed to cover large portions of the body.

In recent years, latex based paints have become common to use for body paint. Latex is a rubber based compound. It is liquid at room temperature, but dries when exposed to air. Liquid latex body paint can be painted on, and then will dry as a thin layer.

There are a number of problems associated with latex based body paints. Most latex paints use an ammonia derivative, and ammonia smells somewhat like urine. As a result there is generally an unpleasant odor associated with latex based body paints. Another problem is that latex based paints can stick to itself, even when dry. This means that there can be problems if significant portions of the body are painted. For example if the torso and arms are painted, the painted person must take care not to let the arms touch the torso. If this happens, the paint will stick to itself and peel off the body when the person moves. Another common problem with latex based paints is that while in the liquid form it will adhere to and stain most cloth material. Another drawback is that in many cases, latex based body paints will fall apart when removed from the body. That means that the latex material won't always come off in one piece, and instead comes off in small clumps, making it difficult to remove. But perhaps the most significant drawbacks to latex based body paints is that a portion of the population is allergic to latex.

The present invention solves many of these problems by disclosing a non-toxic synthetic body paint that generally peels off easily, and that has a pleasant smell or aroma.

### BRIEF SUMMARY OF THE INVENTION

The present invention provides a synthetic, non-toxic, body paint that is easy to remove.

The body paint of the present invention is essentially odorless, and in at least one embodiment, fragrances are added to produce a pleasing aroma.

In at least one embodiment of the invention, the body paint contains pigments, dyes or colored additives to give the paint a variety of colors and visual effects.

In another embodiment of the invention, phosphorescent pigment is added to the body paint to make it glow in the dark.

In another embodiment of the invention, photosensitive pigment is added to the body paint so that it is sensitive to UV—ultraviolet—light.

### BRIEF DESCRIPTION OF THE SEVERAL VIEWS OF THE DRAWINGS

Not Applicable. No drawings.

### DETAILED DESCRIPTION OF THE INVENTION

Detailed embodiments of the present invention are disclosed herein. It is to be understood, however, that the disclosed embodiments are merely exemplary of the invention that may be embodied in various and alternative forms. Therefore, specified structural and functional details disclosed herein are not to be interpreted as limitations, but merely as a basis for the claims and as a representative basis for teaching one skilled in the art to variously employ the present invention.

The present invention is synthetic non-toxic, body paint that is designed to easily peel off the body. The body paint is essentially odorless, and in the preferred embodiment, fragrances are added to provide a body paint with a pleasant aroma.

The disclosed body paint is a liquid solution that is non-toxic and can be safely applied to any part of the body except the eyes and mouth. It can be used by professionals in theatre, sports fans, and people involved in entertainment and recreation. The body paint of the present invention is liquid at room temperatures. It can be applied to the body and will dry into a thin rubber like covering in a matter of minutes. After it dries, it will cling to the body and then can be easily peeled off after use. It leaves no residue. After peeling off, no traces of the body paint will remain. In a preferred embodiment, the addition of fragrances produces a body paint with a pleasant aroma. With the addition of pigments and dyes, it is available in a wide variety of colors, and is also available in invisible hues that glow under blacklight, and others that glow in the dark.

Typically, paints are comprised of a number of components. The four components common to all paints are: (1) pigment, (2) binder, (3) diluent (commonly referred to as thinner) and (4) additives.

Typical pigments include dyes or powderized coloring agents. Generally pigments are made from finely ground and insoluble dispersed particles, made from natural or synthetic, inorganic or organic materials. When the pigments are dispersed in a liquid vehicle (a combination of binders and diluents) they provide color to the paint. The distinction between dyes and powdered pigments is that dyes are soluble, or in solution, when used, and pigments are insoluble and dispersed in the material. Powdered pigments may also, in addition to color, provide other essential properties of the paint such as opacity, hardness, and durability.

The binder is the only component of paint that is absolutely required. Binder is the part of the overall paint which eventually solidifies to form the dried paint film. The binder cements the pigment particles into a uniform paint film and also makes the paint adhere to the surface. Typical binders include synthetic or natural resins such as acrylics, polyurethanes, polyesters, melamines, dextrin, natural and synthetic gums, starch solutions, oils, or latex.

The diluent or thinner serves to adjust the viscosity of the paint. It is volatile and does not become part of the paint film. After application the diluent evaporates allowing the paint to solidify, or in body paints, to partially solidify. Typical diluents or thinners include organic solvents such as alcohols, ketones, esters, glycol ethers, and the like. Water is a common diluent. Sometimes volatile low-molecular weight synthetic resins also serve as diluents.

There are a wide variety of additives that are added to paints. Typical additives include catalysts, thickeners, stabilizers, emulsifers, texturizers, adhesion promoters, flatteners, pH buffers or water softeners, and preservatives. In body paint, plasticizers are commonly added.

Another common additive is a pigment extender or stabilizer, which is commonly a silica (or diatomaceous silica or silica clay), barium sulfate or calcium carbonate. Diatomaceous silica is chemically prepared silicon dioxide.

One additive present in body paints that is not commonly present in other forms of paint is a plasticizer. The plasticizer helps make the body paint soft and porous. Common plasticizers include glucose and glycerine products that help control the absorption of moisture from the air, and prevent the final product from fully solidifying. Other suitable plasticizers include propylene glycol, ethylene glycol, invert sugar, and fructose syrup.

Most paints, particularly those that are maintained in liquid form, contain a preservative. Common preservatives include glutaraldehyde, benzoic acid, para—chloro—meta—xylenol and other phenolic derivatives. Various types of parebens are the most common preservatives for body paint.

Also frequently present in paints that are commonly used in household crafts or used by children are liquid proteins. The pigments more readily bind to the liquid proteins, which helps to prevent staining. Common liquid proteins include a casein solution (derived from cow milk), keratin solution, and albumin solutions.

Many paints include an opacifier, which makes the paint opaque rather than translucent. Common opacifiers include zinc oxide, titanium dioxide, lithopone, and barium sulfate. As can be appreciated, varying levels of opacifier is added to achieve varying painting techniques.

In all forms of paint, including body paint, the paint is applied in liquid form, and after application, the paint hardens. Body paint differs from other forms of paint in that the paint does not fully harden. Instead, due to the presence of the plasticizers, it becomes plastic or semi-solid, with a rubbery feel. This prevents cracking, and allows the paint to move with the body.

Depending on the type of binder used in the paint, the hardening may be a result of curing (polymerization), evaporation, or even cooling. For example, in oil-based paints (which are not used for body paint) curing takes the form of oxidation, as for example the oxidation of linseed oil to form linoxin to create a varnish. In other types of paints, including those commonly used for body paints, when the diluent evaporates, the molecules of the binder coalesce to form a solid, semi-solid, or plastic film.

Non-toxic substances are those that generally are not toxic or harmful to humans. Non-toxic paints are those that have no, or almost no, Volatile Organic Compounds (VOCs). VOC's are present in both the diluent and the binders in many toxic paints. Most of the non-toxic paints use water as the primary diluent, rather than a petroleum based diluent. Non-toxic paints also use a variety of synthetic or natural products for binders.

Acrylic paints are non-toxic paints that use water as the diluent, and acrylic resin as the primary binder. Generically, acrylic resins are resins resulting from the polymerization of derivatives of acrylic acids, including esters of acrylic acid, methacrylic acid, acrylonitrile, and their copolymers. They are often known as acrylate resins. Acrylic Latex Paint is a water-thinned paint which employs acrylic resin as the majority of the binder.

There are a number of natural products that are used as binders in non-toxic paint. These can include natural and synthetic waxes, natural and synthetic gums, starch, gelatin, and a variety of proteins. Beeswax, for example, is one of the oldest known binders for pigment, and creates a paint known as encaustic paint. Other types of wax such as carnauba wax can also be used with the beeswax.

Casein, a milk-based protein product, forms a strong glue when mixed with an alkali (for example lime, borax, or ammonia). Casein paint has a very dry, velvety surface which is rich in color. Casein is water soluble, but dries water insoluble.

Egg tempera paint is made using egg yolk as the binder. The egg yolk also acts as an emulsifier. Synthetic tempera paints are another name for Gouache paints, which use gum arabic acts as the binder. Gum arabic is produced by several species of acacia trees. Guar gum (also called guaran) is also occasionally used in synthetic tempera paints. Gouache differs from water color by the addition of chalk which allows the paint to be more opaque and imparts a dusty quality to the surface. Occasionally oxgall (a wetting agent) is added to the gauche or water color to add in the even dispersion of pigment.

Peel masque products are a type of glycol based skin cleanser that is applied and then peeled off. Typically the peel masque product is applied to the face in a highly viscous form, and allowed to sit for an extended period of time. In some types of peel masque products the alcohol evaporates, allowing the masque to partially solidify. In some types of peel masque products, the masque either heats up or cools off during the drying process. These effects are caused by the addition of various additives, but these types of additives are not appropriate for use in the present invention.

Peel masque products are generally made from glycol based products, and typically also include water, polyvinyl alcohol, denatured alcohol (or denatured ethyl alcohol), as well as fragrances and preservatives. Many masque products also include colorants, cleansers and moisturizers. The addition of cleansers and moisturizers are common in therapeutic masque products, but is not appropriate for use with the present invention.

The glycol based products common in peel masque products are a form of plasticizer, which allows this type of masque product to retain its soft feel and texture. Plasticizers commonly used in peel masque products include glycerine, propylene glycol, polyethylene glycol, and ethoxylated lanoline alcohols. Additionally, alkoxylated long chain alcohols may be used as plasticizers, as well as carboxylilc acids and esters.

The body paint of the present invention is made from the following ingredients. Water, denatured alcohol, polyvinyl alcohol, propylene glycol, non-toxic paint, Oleth-20 (an organic perfume stabilizer derived from unsaturated fatty acids) fragrance, preservatives, and pigment.

The synthetic base of the disclosed body paint is essentially odor free, and is sufficiently stable so that the addition of perfumes or fragrances will not alter the drying or adhering properties of the body paint. Suitable fragrances can include any fragrances approved for cosmetic use. Fragrances commonly used with the disclosed invention include, but are not limited to, Cucumis Sativus (Cucumber) Fruit Extract, Chamomilla Recutita (Matricaria) Extract, Mangifera Indica (Mango) Fruit Extract, Asperigillus Ferment, Ethoxydiglycol, Glycyrrhiza Glabra (Licorice) Root Extract. Many other perfume or fruit extract fragrances can also be used with this product.

Preservatives are commonly used in cosmetics to prevent decay during storage, but they also prevent the build up of molds or funguses when applied to the body. Preservatives used in the present invention include Benzophenone-4, Disodium EDTA, Methylparaben, Butylparaben, Ethylparaben, Isobutylparaben, Propylparaben, Iodopropynyl Butylcarbamate, Phenoxyethanol, and Diazolidinyl UREA.

With the addition of colorants such as pigments and dyes, the body paint will come in a variety of standard colors, such as red, yellow, blue, green, purple, orange, pink, light green, and light blue. It is within the scope of the present invention that with the addition of the appropriate pigment or dye, the body paint can be produced in any color of the visible color spectrum. Colorants that can conceivably be used include all of the FD&C colors approved for cosmetic use, as well as various organic and inorganic pigments, vegetable dyes and other naturally occurring earth pigments. Common pigments include indigo, ocher, umber, carbon clack, iron oxide, D & C Yellow No. 5, Clarion Red, Barium Lithol, D & C Blue No. 1, D & C Red No. 27, Victoria Blue Lake, Prussian Blue, Lithol Rubine, D & C Yellow No. 6, FD & C Blye No. 2, Acid Violet 4 BNS, Calcium Lithol and Titanium Dioxide.

The addition of reflective metal oxide mica will create glitter body paint with a glitter effect. Pigments and dyes can be added to the glitter paint to create a variety of colors, such as silver, gold, red, purple, blue, green, yellow, and orange.

With the addition of a phosphorescent or photo-luminescent pigment, a glow in the dark effect can be achieved. Phosphorescent or photo-luminescent pigments are typically made from alkaline earth metal aluminate, where the earth metals can include strontium, magnesium, calcium, and barium. Silicon and titanium may also be present as a stabilizer.

Fluorescent effects can be created with the body paint of the present invention with the addition of fluorescent pigment.

Ultra-violet pigment can also be added to the body paint to produce ultra-violet or “black light” sensitive body paints.

In at least one embodiment of the present invention, the invention is produced by mixing synthetic tempera paint with peel masque.

Typically tempera paint is not suitable for use as body paint because it is far too liquid and runny, and will run off the body before it fully dries. It also takes a long time to dry, often as much as an hour or longer. And when it does fully dry it tends to be too solid. It does not move with the body, and often flakes off. This also makes it difficult to remove.

The process of fusing the synthetic tempera paint with the glycol based peel masque produces a product that is suitable for use as a body paint. The body paint dries quickly, generally in about 5 minutes, due to the alcohol in the masque. The body paint adheres easily to the skin, due to the propylene glycol. The body paint also peels off easily, and leaves virtually no traces of color on the skin.

The present invention is well adapted to carry out the objectives and attain both the ends and the advantages mentioned, as well as other benefits inherent therein. While the present invention has been depicted, described, and is defined by reference to particular embodiments of the invention, such reference does not imply a limitation to the invention, and no such limitation is to be inferred. The depicted and described embodiments of the invention are exemplary only, and are not exhaustive of the scope of the invention. Consequently, the present invention is intended to be limited only be the spirit and scope of the claims, giving full cognizance to equivalents in all respects.

I claim:

1. A synthetic peel off body paint comprising
a non-toxic paint, and
a peel masque.

2. The synthetic peel off body paint of claim 1, wherein said peel masque is comprised of a plasticizer and water.

3. The synthetic peel off body paint of claim 2, wherein said plasticizer is selected from the group consisting of glycerine, propylene glycol, polyethylene glycol, and ethoxylated lanoline alcohol.

4. The synthetic peel off body paint of claim 1, wherein said non-toxic paint is comprised of:
a binder;
a pigment; and
a diluent.

5. The synthetic peel off body paint of claim 4, wherein said binder is selected from the group consisting of natural waxes, synthetic waxes, natural gums, synthetic gums, starch, gelatin, casein, and egg yolk.

6. The synthetic peel off body pain of claim 4 wherein said binder is selected from the group consisting of gum arabic and guar gaur gum.

7. The synthetic peel off body paint of claim 4, wherein said pigment is selected from the group consisting of organic pigments, inorganic pigments, vegetable dyes and other naturally occurring earth pigments.

8. The synthetic peel off body paint of claim 4, further comprising a reflective metal oxide mica.

9. The synthetic peel off body paint of claim 4, further comprising a photo-luminescent pigment.

10. The synthetic peel off body paint of claim 4, further comprising a fluorescent pigment.

11. The synthetic peel off body paint of claim 4, further comprising an ultra-violet pigment.

12. The synthetic peel off body paint of claim 1 further comprising a fragrance.

13. The synthetic peel off body paint of claim 9, wherein said fragrance is selected from the group consisting of Cucumis Sativus (Cucumber) Fruit Extract, Chamomilla Recutita (Matricaria) Extract, Mangifera Indica (Mango) Fruit Extract, Asperigillus Ferment, and Glycyrrhiza Glabra (Licorice) Root Extract.

14. The synthetic peel off body paint of claim 1 further comprising a preservative.

15. The synthetic peel off body paint of claim 10, wherein said preservative is selected from the group consisting of Benzophenone-4, Disodium EDTA, Methylparaben, Butylparaben, Ethylparaben, Isobutylparaben, Propylparaben, Iodopropynyl Butylcarbamate, Phenoxyethanol, and Diazolidinyl UREA.

16. A method of making a synthetic peel off body paint comprising the steps of:
preparing a non-toxic paint; and
mixing a peel off masque to said non-toxic paint.

17. The method of making a synthetic peel off body paint of claim 16 wherein said non-toxic paint is a synthetic tempera paint.

18. The method of making a synthetic peel off body paint of claim 18 wherein said peel masque includes a plasticizer.

19. The method of making a synthetic peel off body paint of claim 18 wherein said plasticizer is selected from the group consisting of glycerine, propylene glycol, polyethylene glycol, and ethoxylated lanoline alcohol.
